(19) United States
(12) Patent Application Publication
(10) Pub. No.: US 2015/0148551 A1
(43) Pub. Date: May 28, 2015

(54) GENISTEIN FOR REDUCING LEVELS OF STORAGE COMPOUNDS IN THE TREATMENT AND/OR PREVENTION OF LYSOSOMAL STORAGE DISEASES (LSDS)

(71) Applicant: 3G THERAPEUTICS, INC., Miami, FL (US)

(72) Inventors: Grzegorz Wegrzyn, Gdansk (PL); Magdalena Angelina Gabik-Ciminska, Straszyn (PL); Joanna Jakobkiewicz-Banecka, Gdansk (PL); Marta Moskot, Gdynia (PL)

(21) Appl. No.: 14/406,276

(22) PCT Filed: Jun. 7, 2013
(86) PCT No.: PCT/IB2013/001696

§ 371 Date: Dec. 8, 2014

(30) Foreign Application Priority Data
Jun. 8, 2012 (PL) P 399 467

(51) Int. Cl. A61K 31/352

(52) U.S. Cl. CPC A61K 31/352
USPC 549/403

(57) ABSTRACT

The subject of the invention is genistein for use in non-enzymatic method of treatment and/or prevention of the lysosomal storage diseases (LSDs) i.e. diseases with the underlying defect in degradation and resulting accumulation of organic compounds in lysosomes, to reduce the level of storage of organic compounds by reducing the rate of efficiency of accumulating organic substances synthesis and/or by increasing the rate of efficiency of cellular deposits degradation.

The subject of the invention is the use of genistein to reduce levels of storage compounds in the cells of patients with lysosomal storage diseases (LSDs), especially to reduce the rate of synthesis and to increase the rate of degradation of accumulated substances. More specifically, the invention relates to the potential use of genistein in the new non-enzymatic method of lysosomal storage diseases treatment by modulation of expression of genes encoding enzymes of biosynthesis and degradation pathways of organic substances accumulated in the cells of patients with LSD and enzymes which modify these compounds.

Lysosomal storage diseases (LSDs) are a group of over 50 inherited metabolic diseases whose common feature are the disturbances of lysosomal function due to specific deficit in the activity of lysosomal enzymes, transport proteins or lysosomal membrane receptors [Saftig, 2005]. The result of these impairments is a disrupted degradation of specific substances, which accumulate in the lysosomes and in turn cause the dysfunction of cells, tissues and organs by accumulating in the lysosomes. These diseases are characterized by a tendency towards progression. [consider: Usually, LSDs are progressive in nature.] Typically, the symptoms occur within a few months after birth and gradually progress leading, in most cases, to death in childhood or puberty. LSDs are among the first genetic disorders, for which therapy, effective to some extent, became feasible [Beck, 2010]. Additionally, molecular mechanisms of these diseases are rather well known in comparison to other similar disorders, and therefore lysosomal storage diseases can be considered as a good research model in studies of genetically determined metabolic diseases.

Generally, the treatment of the above mentioned LSDs can be divided into enzymatic and non-enzymatic. Currently available enzymatic therapies are bone marrow transplantation and enzyme replacement therapy (ERT) [Lim-Melia i Kronn, 2009]. ERT is based on intravenous infusion of recombinant human enzyme, which is found to be absent or deficient in the cells of the patient. Currently, the ERT is available for only six out of over fifty LSDs, i.e. Gaucher disease, Fabry disease, Pompe disease and three types of mucopolysaccharidosis (MPS I, II and VI). Unfortunately, the ERT is not a perfect therapeutic method. This is partly because none of the currently available recombinant enzymes used in ERT, which can be delivered to the most of tissues and organs, has the ability to cross the blood-brain barrier (BBB) and therefore it does not affect neurological symptoms, which repeatedly come to the fore in the clinical picture of the majority of LSDs [Rohrbach i Clarke, 2007]. Additionally, ERT is not always able to improve or inhibit the progression of the disease in all of the involved organs or systems. Moreover, similarly to most therapeutic methods, ERT has many adverse effects, the most important of which are serious anaphylactic reactions after the use of a medication. Regarding gene therapy, although it may offer a hope for patients with LSD, the treatment with this method is still in the research phase [Beck, 2010; Przemyśl i Haskins, 2007]. Therefore, there is still a need for alternative therapies, which could be helpful for patients suffering from lysosomal storage diseases, especially those with neuronopathic disorders.

In Patent Application P-390766 (publ. Sep. 26, 2011) complex C-glycosides, genistein derivatives having cytotoxic and anti-proliferative activity, and their application is described. The invention discloses complex C-glycosides, genistein derivatives having the structure of formula 1, wherein the sugar unit, which is per-O-acylated mono-di- or trisaccharide, 2,3-polyunsaturated mono-, di- or trisaccharide, is joined by the C-glycosylic bond with the linker comprising from two up to ten carbon atoms, in which the functional A group is a double bond, ether, thioether, ester, thioester, amide, thioamide, acetal, thioacetal group, and the substituents in the linker are halogen, alkoxy, alkoxycarbonyl, heteraryl, hydroxyl, hydroxyalkyl, hydroxyaryl, alkylamine, dialkylamine group, and a linker is joined by 7-O-etheric bond with genistein. Disclosed is the use of complex C-glycosides, genistein derivatives having cytotoxic and anti-proliferative activity, for manufacturing of an agent intended for prevention and treatment of cancer, which is a preparation having anti-cancer activity.

In Patent Application P-367929 (publ. Nov. 14, 2005) the new application of genistein derivatives is described. The invention discloses new application of genistein and derivatives thereof of formula I, wherein: at least one R₁ or R₂ group is alkyl, alkenyl, aryl, alkylaryl, alkylcarbonyl, arylcarbonyl or mono-, di- or oligosaccharide group, in which hydroksyl groups are optionally substituted with the same or different acyl, alkyl, acyloxyalkyl or aryl groups, while the other R₁ or R₂ group has the same or different meaning or is a hydrogen atom, R₃ is a hydrogen atom or COCH₃ group and pharmaceutically acceptable salts thereof for manufacturing of pharmaceutical agent having analgesic activity.

In Patent Specification PL 205 635 (publ. Oct. 21, 2002) genistein derivatives of formula I are shown, wherein R₁ and R₂ are the same or different and independently represent a hydrogen atom, alkyl, allyl, aryl, alkylaryl, alkylcarbonyl, arylcarbonyl, wherein each of the above group may be substituted, R₅(R₆)R₇—Si— group, wherein R₅, R₆ and R₇ are the same or different and represent C1-6 alkyl or aryl or R₁ and R₂ represent mono-, di- or oligosaccharide group, wherein at least one hydroxyl group of saccharide group may be substituted with the same or different acyl, alkyl, acyloxyalkyl or aryl groups; R₃ is hydrogen atom or —COCH₃ group; and R₄ is hydrogen atom, —SO₃H, SO_{3<̂>—}, —NH₂ or —NO₂ group, and pharmaceutically accepted salts thereof are applicable for manufacturing of a pharmaceutical agent having anti-cancer activity.

Patent Application US 2010204162 (publ. Aug. 12, 2010) describes the treatment consisting of the use of compounds according to the principle for reduction of substrates. The invention provides a compound being a sphingolipid biosynthesis inhibitor for the use in the treatment of Niemann-Pick disease type C.

Although the molecular mechanisms of lysosomal storage diseases are rather well known, in comparison to other similar disorders, and although they can be considered as research models in the studies on inherited metabolic diseases, there is a ongoing need for developing solutions enabling their treatment.

In the search of new opportunities for lysosomal storage diseases treatment in the previous studies the inventors of the invention found, that genistein (4′,5,7-trihydroxy-3-phenylchromen-4-one), a compound from the group of isoflavones, which are naturally occurring in many plants (especially abundantly in soybeans), inhibits the synthesis of glicosaminoglicanes (GAG)—substances, which accumulate in the cells of the patients with mucopolysaccharidosis (MPS), due to dysfunction of one of the lysosomal enzymes [Neufeld i Muenzer, 2001; Piotrowska et al., 2006]. Because the GAG degradation in these cells is ineffective, the inventors presumed, that lowering their level of synthesis could lead to the re-establishing of a balance between their production and degradation. The validity of this assumption was proved by the results of the studies of the inventors, carried out with the use of fibroblasts from patients with MPS type I, IIIA and IIIB, which showed, that genistein reduces levels of GAG synthesis, and moreover, it causes disappearance of the storage depositions in cells [Piotrowska i wsp., 2006]. Very important assets of genistein are that this compound is well-tolerated by humans and animals, and it has the ability to cross the blood-brain barrier [Tsai, 2005]. This latter feature offers hope for the treatment of neurodegenerative forms of LSD, including MPS.

More precise studies on the cellular level have allowed to discover molecular basis of genistein activity, while animal tests and pilot clinical trials have shown promising effectiveness in the treatment of MPS II and III [Malinowska i wsp., 2009; Friso i wsp., 2010].

The aim of the studies carried out recently was to access the possibility to use the new, non-enzymatic method of treatment of lysosomal storage diseases with the use of genistein to lower the levels of accumulated organic substances by modulating gene expression encoding lysosomal hydrolases, and also GAG synthetases and enzymes modifying their chains. Based on the data from literature, as well as the results of the studies of the inventors it was recognized, that genistein can inhibit GAG synthesis due to disturbances in the expression of genes encoding one or more of the enzymes involved in this process by inhibiting the tyrosine kinase activity of epidermal growth factor receptor (EGF) [JakObkieiwcz-Banecka et al., 2009]. Due to the implied mechanism of genistein action, the inventors suggested the name for the ‘gene-expression targeted isoflavone therapy’ (GET IT). However it should be noted, that to date, molecular mechanism of GET IT therapy with the use of genistein remains unexplained.

The aim of the studies, which had led to the invention, was the investigation of molecular mechanism of genistein action, especially the issues regarding transcriptome profiling of cells exposed to genistein. The object of the studies was to investigate the role of this isoflavone in the regulation of the expression of genes involved in the metabolism of the substances accumulated in the cells of patients with LSD: 1) by lowereing expression of the genes involved in the substrate synthesis—substrate synthesis reducing therapy, SRT; and/or 2) by increasing expression of the genes involved in the degradation of the cellular deposits.

The subject of the invention is genistein for use in non-enzymatic method of treatment and/or prevention of the lysosomal storage diseases (LSDs) i.e. diseases with the underlying defect in degradation and resulting accumulation of organic compounds in lysosomes, to reduce the level of storage of organic compounds by reducing the rate of efficiency of accumulating organic substances synthesis and/or by increasing the rate of efficiency of cellular deposits degradation.

Preferably the increase of the efficiency of degradation of the organic substances accumulated in the cells of patients with LSD exposed to genistein, due to the modulation of expression of genes encoding one or more of the enzymes involved in this process, occurs by the overproduction of the transcription factor EB (TFEB) due to increased expression of its gene.

Preferably the decrease of the efficiency of synthesis of the organic substances accumulated in the cells of patients with LSD exposed to genistein is due to the modulation of expression of genes encoding one or more of the enzymes involved in this process.

Preferably there is a modulation of expression of genes involved in the metabolism of storage substances (i.e. synthesis and degradation) i.e. genes encoding for enzymes of the biosynthesis pathway of organic substances accumulating in cells of patients with LSD and enzymes, which are modifying these compounds, as well as lysosomal enzymes.

Preferably gene expression is monitored with the use of transcriptomic methods.

Preferably DNA microarrays are used.

Preferably the impaired enzymatic activity of lysosomes relates to enzymes selected from alpha-L-iduronidase, N-sulfoglucosamine sulfohydrolase, alpha-D-N-acetylglucosaminidase, alpha-N-acetylglucosamine-6-sulphate sulfatase, beta-hexosaminidase A, hyaluronglucosaminidase 3, alpha-glucosidase, beta-glucosidase, alpha-fucosidase, alpha-mannosidase, beta-mannosidase, sialidase 1, GM2 activator, beta-hexosaminidase A, N-acylsphingosine aminohydrolase, sphingomyelin phosphodiesterase 1, Niemann-Pick C1 protein, tripeptidyl peptidase 1, neuronal ceroid lipofuscinose protein 5, UDP-N-acetylglucosamine-1-phosphotransferase, mucolipin 1, cystinosin, sulfatase modifying factor 1, aspartylglucosaminidase, arylsulfatase A, arylsulfatase G, cathepsin A, cathepsin D, cathepsin F, cathepsin K, cathepsin O, legumin, acid phosphtase 2, acid phosphtase 5, hydrosphingosine reductase, factor 9 of subfamily A (ABC 1) of proteins containing an ATP binding cassette, sialomucins, proton-dependent divalent metal transporter. Furthermore, the use according to claim 1, wherein the reduced level of biosynthesis of organic substances accumulated in the cells of patients with LSD and modifying these compounds relates to enzymes selected from: N-acetylgalactosamine sulfotransferase, glucosamine sulfotransferase 3A1, N-acetylglucosamine transferase, xylosyltransferase and alpha-sialyltransferases 2, 4 and 6.

Preferably the disease entity is selected from MPS I, MPS IIIA, MPS IIIB, MPS IIID, Pompe disease, Gaucher disease, fucosidosis, alpha-mannosidosis, beta-mannosidosis, sialosis/sialadenosis/galactosialosis, GM2 gangliosidosis type AB, GM2 gangliosidosis type I (Tay-Sachs disease), Farber disease, Niemann-Pick disease type A and B, Niemann-Pick disease type C, neuronal ceroid lipofuscinose type II, neuronal ceroid lipofuscinose type V, mucolipidosis type II and III A and B, mucolipidosis type IV, cystinosis, mucosulfatidosis, aspartylglucosoaminuria, metachromatic leukodystrophy, pycnodysostosis and other selected disease entities from lysosomal storage diseases.

For a better illustration of the invention, the solution is shown on the drawing, where: FIG. 1 shows the results of the expression studies of (A) genes from biosynthesis pathway of organic substances accumulated in the cells of patients with LSD, and (B) genes coding for lysosomal hydrolases involved in the metabolism of substrates, which storage in cells is responsible for certain LSD. The individual mRNA levels were measured after 24 and 48 hours of cell exposition to 100 μM genistein. The bars represent the mean change in the expression±SD, n=5 and comprise statistical variables (significance level of p<0.1) between the level of individual transcripts in the test sample to the control sample, in comparison to reference genes GAPDH and TBP, showing constant levels of expression.

Examples below illustrate the subject invention.

### EXAMPLE 1

### Microarray Analysis of Human Fibroblast Genome in Response to Genistein Treatment

The experiments were conducted to characterise the transcriptomic profile of human fibroblast cells exposed to genistein, and in particular to determine the expression of genes encoding lysosomal enzymes involved in metabolism (i.e. synthesis and degradation) of organic substances. The absence or deficiency of these enzymes' activity is responsible for occurrence of various lysosomal storage diseases (LSD) (including MPS). The tested hypothesis assumed that genistein influences the expression of many genes, among which there are genes coding for enzymes required in metabolism of compounds, which are pathologically stored in LSD. The results provide important information regarding genistein action on the molecular level within the range of gene expression modulation by this substance. The experiment was conducted in biological quintuplicates. Human dermal fibroblasts (HDFa) were exposed in vitro for 1, 24 or 48 hours to: 30, 60 or 100 μM of genistein; 0.05% DMSO (K1); or were untreated (control cells) (K2), and total RNA was isolated and analysed.

### Culture Conditions

Fibroblast cells were grown in Dulbecco (DMEM) medium supplemented with 10% fetal bovine serum (FBS) with the addition of 1% antibiotics and anti-fungal agents at 37° C. in humidified atmosphere of 5% CO₂, until the required confluency was obtained.

### Test Conditions

To determine the effect of tested compound on the transcriptom of the fibroblast cells, the growth medium was replaced with fresh medium, either non-supplemented (0.05% DMSO), or containing genistein (30, 60 or 100 μM, in 0.05% DMSO). The exposure was done for 1, 24 or 48 hours.

### Extraction Protocol

Total RNA was isolated from fibroblasts using High Pure RNA Isolation Kit according to the protocol and then it was quantitatively evaluated using Quant-It™ RiboGreen® Assay Kit.

### Labelling Protocol

Biotinylated cRNA was prepared using Ambion Total PrepAmp Kit for Illumina arrays.

### Hybridization and Scanning Protocol

Standard hybridization and quantitative image analysis procedures—Illumina hybridization protocol and Illumina scanning protocol, were used.

### Data Processing and Definition

Processing of the results was done during data definition, including: background correction (using background subtraction method), normalization (using quantile normalization algorithm with Illumina GenomeStudio software package: gene expression module version 1.9.0) and summarization (using method, which sums the values of signal intensities from a set of probes).

### Biological

### Samples

| Name | | |
|---|---|---|
| of the | | |
| biological | | Biological |
| sample | Description of the biological sample | replicate |
| 1h30_1 | fibroblast_30 μM genistein_1h_rep1 | replicate 1 |
| 1h100_1 | fibroblast_100 μM genistein_1h_rep1 | replicate 1 |
| 1hK1_1 | fibroblast_K1_0.05%DMSO_1h_rep1 | replicate 1 |
| 1hK2_1 | fibroblast_K2_untreated_1h_rep1 | replicate 1 |
| 24h30_1 | fibroblast_30 μM genistein_24h_rep1 | replicate 1 |
| 24h100_1 | fibroblast_100 μM genistein_24h_rep1 | replicate 1 |
| 24hK1_1 | fibroblast_K1_0.05%DMSO_24h_rep1 | replicate 1 |
| 24hK2_1 | fibroblast_K2_untreated_24h_rep1 | replicate 1 |
| 48h30_1 | fibroblast_30 μM genistein_48h_rep1 | replicate 1 |
| 48h100_1 | fibroblast_100 μM genistein_48h_rep1 | replicate 1 |
| 48hK1_1 | fibroblast_K1_0.05%DMSO_48h_rep1 | replicate 1 |
| 48hK2_1 | fibroblast_K2_untreated_48h_rep1 | replicate 1 |
| 1h30_2 | fibroblast_30 μM genistein_1h_rep2 | replicate 2 |
| 1h100_2 | fibroblast_100 μM genistein_1h_rep2 | replicate 2 |
| 1hK1_2 | fibroblast_K1_0.05%DMSO_1h_rep2 | replicate 2 |
| 1hK2_2 | fibroblast_K2_untreated_ 1h_rep2 | replicate 2 |
| 24h30_2 | fibroblast_30 μM genistein_24h_rep2 | replicate 2 |
| 24h100_2 | fibroblast_100 μM genistein_24h_rep2 | replicate 2 |
| 24hK1_2 | fibroblast_K1_0.05%DMSO_24h_rep2 | replicate 2 |
| 24hK2_2 | fibroblast_K2_untreated_24h_rep2 | replicate 2 |
| 48h30_2 | fibroblast_30 μM genistein_48h_rep2 | replicate 2 |
| 48h100_2 | fibroblast_100 μM genistein_48h_rep2 | replicate 2 |
| 48hK1_2 | fibroblast_K1_0.05%DMSO_48h_rep2 | replicate 2 |
| 48hK2_2 | fibroblast_K2_untreated_48h_rep2 | replicate 2 |
| 1h30_3 | fibroblast_30 μM genistein_1h_rep3 | replicate 3 |
| 1h100_3 | fibroblast 60 μM genistein_1h_rep3 | replicate 3 |
| 1hK1_3 | fibroblast_100 μM genistein_1h_rep3 | replicate 3 |
| 1hK2_3 | fibroblast_K1_0.05%DMSO_1h_rep3 | replicate 3 |
| 24h30_3 | fibroblast_30 μM genistein_24h_rep3 | replicate 3 |
| 24h100_3 | fibroblast_60 μM genistein_24h_rep3 | replicate 3 |
| 24hK1_3 | fibroblast 100 μM genistein_24h rep3 | replicate 3 |
| 24hK2_3 | fibroblast_K1_0.05%DMSO_24h_rep3 | replicate 3 |
| 48h30_3 | fibroblast_30 μM genistein_48h_rep3 | replicate 3 |
| 48h100_3 | fibroblast 60 μM genistein_48h_rep3 | replicate 3 |
| 48hK1_3 | fibroblast_100 μM genistein_48h_rep3 | replicate 3 |
| 48hK2_3 | fibroblast_K1_0.05%DMSO_48h_rep3 | replicate 3 |
| 1h30_4 | fibroblast_30 μM genistein_1h_rep4 | replicate 4 |
| 1h100_4 | fibroblast 60 μM genistein_1h_rep4 | replicate 4 |
| 1hK1_4 | fibroblast 100 μM genistein_1h_rep4 | replicate 4 |
| 1hK2_4 | fibroblast_K1_0.05%DMSO_1h_rep4 | replicate 4 |
| 24h30_4 | fibroblast_30 μM genistein_24h_rep4 | replicate 4 |
| 24h100_4 | fibroblast_60 μM genistein_24h_rep4 | replicate 4 |
| 24hK1_4 | fibroblast 100 μM genistein_24h_rep4 | replicate 4 |
| 24hK2_4 | fibroblast_K1_0.05%DMSO_24h_rep4 | replicate 4 |
| 48h30_4 | fibroblast_30 μM genistein_48h_rep4 | replicate 4 |
| 48h100_4 | fibroblast_60 μM genistein_48h_rep4 | replicate 4 |
| 48hK1_4 | fibroblas_100 μM genistein_48h_rep4 | replicate 4 |
| 48hK2_4 | fibroblast_K1_0.05%DMSO_48h_rep4 | replicate 4 |
| 1h30_5 | fibroblast_30 μM genistein_1h_rep5 | replicate 5 |
| 1h100_5 | fibroblast_60 μM genistein_1h_rep5 | replicate 5 |
| 1hK1_5 | fibroblast_100 μM genistein_1h_rep5 | replicate 5 |
| 1hK2_5 | fibroblast_K1_0.05%DMSO_1h_rep5 | replicate 5 |
| 24h30_5 | fibroblast_30 μM genistein_24h rep5 | replicate 5 |
| 24h100_5 | fibroblast_60 μM genistein_24h_rep5 | replicate 5 |
| 24hK1_5 | fibroblast_100 μM genistein_24h_rep5 | replicate 5 |
| 24hK2_5 | fibroblast_K1_0.05%DMSO_24h_rep5 | replicate 5 |
| 48h30_5 | fibroblast_30 μM genistein_48h_rep5 | replicate 5 |

### Data on Expression of Reference Genes GAPDH and TBP-Fold Change.

| | Average | SD | Average | SD | Average | SD |
|---|---|---|---|---|---|---|
| SYMBOL | 1h30/1hK1 | 1h30/1hK1 | 1h60/1hK1 | 1h60/1hK1 | 1h100/1hK1 | 1h100/1hK1 |
| GAPDH | 0.9591955 | 0.0550866 | 0.9816665 | 0.0303501 | 0.93852394 | 0.03641494 |
| TBP | 0.9739844 | 0.1515444 | 0.9182535 | 0.1055156 | 0.95309397 | 0.18254314 |
| | Average | SD | Average | SD | Average | SD |
| SYMBOL | 24h30/24hK1 | 24h30/24hK1 | 24h60/24hK1 | 24h60/24hK1 | 24h100/24hK1 | 24h100/24hK1 |
| GAPDH | 0.929988697 | 0.060038421 | 0.89050624 | 0.078021918 | 0.955641184 | 0.075098736 |
| TBP | 0.881604521 | 0.040387118 | 0.789182931 | 0.10909342 | 0.830299777 | 0.104111198 |
| | Average | SD | Average | SD | Average | SD |
| SYMBOL | 48h30/48hK1 | 48h30/48hK1 | 48h60/48hK1 | 48h60/48hK1 | 48h100/48hK1 | 48h100/48hK1 |
| GAPDH | 0.941153686 | 0.024487477 | 0.808760157 | 0.058502876 | 0.865188913 | 0.10046261 |
| TBP | 0.926530142 | 0.126785203 | 0.992646273 | 0.107373013 | 0.886249887 | 0.0953391 |

Data on the Expression of Genes Encoding Enzymes of Synthesis Pathways of Organic Substances Accumulated in the Cells of Patients with LSD—Fold Change.

| | Average | SD | Average | SD | Average | SD |
|---|---|---|---|---|---|---|
| SYMBOL | 1h30/1hK1 | 1h30/1hK1 | 1h60/1hK1 | 1h60/1hK1 | 1h100/1hK1 | 1h100/1hK1 |
| CHST14 | 1.007108 | 0.269512 | 0.851032 | 0.069926 | 1.060017 | 0.204824 |
| EXT1 | 0.91382 | 0.164235 | 0.988124 | 0.178522 | 0.835841 | 0.13128 |
| HS3ST3A1 | 1.026844 | 0.160443 | 0.900194 | 0.088027 | 1.047142 | 0.165373 |
| ST3GAL2 | 1.154646 | 0.317656 | 1.466614 | 0.107211 | 1.338462 | 0.343797 |
| XYLT1 | 1.057576 | 0.193925 | | | 1.000408 | 0.135185 |
| ST3GAL4 | 0.988476 | 0.19618 | 0.920492 | 0.235468 | 0.863652 | 0.20564 |
| ST3GAL6 | 1.233572 | 0.0916 | 1.071823 | | 1.063686 | 0.082252 |
| | Average | SD | Average | SD | Average | SD |
| SYMBOL | 24h30/24hK1 | 24h30/24hK1 | 24h60/24hK1 | 24h60/24hK1 | 24h100/24hK1 | 24h100/24hK1 |
| CHST14 | 0.823377 | 0.065817 | 0.818446 | 0.098942 | 0.843812 | 0.357135 |
| EXT1 | 0.804791 | 0.238225 | 0.700489 | 0.175625 | 0.602217 | 0.19168 |
| HS3ST3A1 | 0.834997 | 0.129103 | 0.574444 | 0.043404 | 0.545157 | 0.158763 |
| ST3GAL2 | 0.75051 | 0.130278 | 0.506825 | 0.142149 | 0.629895 | 0.105736 |
| XYLT1 | 0.86106 | 0.085159 | 0.621664 | | 0.632419 | 0.166828 |
| ST3GAL4 | 0.781987 | 0.161163 | ST3GAL4 | 0.920458 | 0.664585 | 0.153851 |
| ST3GAL6 | 0.9878 | | ST3GAL6 | | 0.737064 | |
| | Average | SD | Average | SD | Average | SD |
| SYMBOL | 48h30/48hK1 | 48h30/48hK1 | 48h61/48hK1 | 48h60/48hK1 | 48h100/48hK1 | 48h100/48hK1 |
| CHST14 | 0.921749 | 0.274131 | 0.852611 | 0.184918 | 0.653358 | 0.111582 |
| EXT1 | 0.929097 | 0.132457 | 0.764772 | 0.09085 | 0.688967 | 0.101484 |
| HS3ST3A1 | 0.793286 | 0.110745 | 0.604259 | 0.071702 | 0.568959 | 0.124332 |
| ST3GAL2 | 0.796986 | 0.153043 | | | 0.721462 | 0.005404 |
| XYLT1 | 0.974477 | 0.083969 | | | 0.921042 | 0.106044 |
| ST3GAL4 | 1.064932 | 0.217558 | 0.993094 | 0.25622 | 0.891075 | 0.183626 |
| ST3GAL6 | 1.015157 | | | | 0.74085 | |

### Data on Expression of Genes Encoding Lysosomal Enzymes—Fold Change.

| | | Average | SD | Average | SD | Average | SD |
|---|---|---|---|---|---|---|---|
| Syndrome | SYMBOL | 1h30/1hK1 | 1h30/1hK1 | 1h60/1hK1 | 1h60/1hK1 | 1h100/1hK1 | 1h100/1hK1 |
| MPS I | IDUA | 0.8853 | 0.1998 | 0.978768 | 0.239319 | 0.903405 | 0.236583 |
| MPS IIIA | SGSH | 1.0703 | 0.0944 | 1.065043 | 0.140301 | 1.069923 | 0.058256 |
| MPS IIIB | NAGLU | 1.055 | 0.1285 | 1.060807 | 0.177005 | 1.036871 | 0.09925 |
| MPS IIID | GNS | 1.0062 | 0.15 | 1.021808 | 0.067994 | 1.0219 | 0.173262 |
| Degradation of GAG/GM2 gangliosidosis type I | HEXA | 1.2226 | 0.5171 | 1.174032 | 0.4163 | 1.041807 | 0.170547 |
| Degradation of GAG | HYAL3 | 1.1887 | 0.3932 | | | 1.140838 | 0.277694 |
| Pompe Disease | GAA | 0.9686 | 0.2908 | 1.086917 | 0.169705 | 0.934994 | 0.286053 |
| Gaucher disease | GBA | 0.9138 | 0.0734 | 1.033408 | 0.014223 | 0.946921 | 0.102258 |
| Fucosidosis | FUCA1 | 0.9984 | 0.0804 | 1.022059 | 0.149854 | 0.994499 | 0.156715 |
| Alpha-mannosidosis | MAN2B1 | 1.0342 | 0.1096 | 0.850696 | 0.095171 | 0.986379 | 0.277048 |
| Beta-mannosidosis | MANBA | 1.0095 | 0.1647 | 1.04076 | 0.130907 | 0.948861 | 0.136338 |
| Sialosis/sialadenosis/galactosialosis | NEU1 | 1.2206 | 0.2324 | 1.322522 | 0.228073 | 1.353356 | 0.198233 |
| GM2 gangliosidosistype AB | GM2A | 1.5655 | | 1.180857 | 0.01903 | 1.254291 | 0.349941 |
| Farber Disease | ASAH1 | 1.1419 | 0.2339 | 1.028799 | 0.149504 | 1.251882 | 0.35835 |
| Niemami-Pick disease type A and B | SMPD1 | 1.0078 | 0.123 | 0.983109 | 0.028751 | 1.011587 | 0.182035 |
| Niemann-Pick disease C | NPC1 | 0.9973 | 0.0723 | 1.03501 | 0.092328 | 0.982506 | 0.080461 |
| Neuronal ceroid lipofuscinose type II | TPP1 | 1.0655 | 0.0668 | 1.000619 | 0.011769 | 1.030544 | 0.036503 |
| Neuronal ceroid lipofuscinose type V | CLN5 | 1.0505 | 0.1783 | 1.028104 | 0.139112 | 1.050181 | 0.116726 |
| Mucolipidosis II and IIIA and B | GNPTAB | 0.7878 | 0.1148 | 1.234663 | | 0.862832 | |
| Mucolipidosis IV | MCOLN1 | 1.065 | 0.2142 | 1.03795 | 0.100048 | 1.012715 | 0.124724 |
| Cystinosis | CTNS | 1.0024 | 0.037 | 0.79975 | | 0.796363 | |
| Mucosulfatidosis | SUMF1 | 1.1314 | 0.1021 | 1.157755 | 0.230601 | 1.095422 | 0.094925 |
| Aspartylglucosaminuria | AGA | 1.0395 | 0.1233 | 0.975298 | 0.147253 | 0.974853 | 0.187333 |
| Metachromatic leukodystrophy | ARSA | 0.9099 | 0.1206 | 0.909243 | 0.083237 | 0.963039 | 0.142833 |
| Sulfatosis (unknown disease entity) | ARSG | 1.083 | 0.2892 | 1.203771 | 0.145266 | 0.960967 | 0.096233 |
| Proteosis (unknown disease entity) | CTSA | 1.0199 | 0.1317 | 0.981584 | 0.129059 | 0.964312 | 0.058367 |
| Proteosis (unknown disease entity) | CTSD | 0.9734 | 0.0962 | 1.11867 | 0.131304 | 1.018437 | 0.094384 |
| Proteosis (unknown disease entity) | CTSF | 1.0096 | 0.1027 | 0.95054 | 0.039006 | 0.945761 | 0.060228 |
| Pycnodysostosis | CTSK | 0.9778 | 0.1172 | 1.108389 | 0.038508 | 0.986838 | 0.135619 |
| Proteosis (unknown disease entity) | CTSO | 0.9973 | 0.2301 | 1.000311 | 0.295855 | 1.069788 | 0.149396 |
| Proteosis (unknown disease entity) | LGMN | 0.9392 | 0.0812 | 0.996252 | 0.02231 | 0.897846 | 0.061775 |
| Phosphatosis (unknown disease entity) | ACP2 | 1.0116 | 0.2244 | 0.94997 | 0.229923 | 0.980418 | 0.177827 |
| Phosphatosis (unknown disease entity) | ACP5 | 0.9865 | 0.0963 | 1.066121 | 0.205372 | 1.140845 | 0.236544 |
| Lipidosis (unknown disease entity) | FVT1 | 1.0953 | 0.1147 | 1.137494 | 0.100648 | 1.035122 | 0.108703 |
| Membrane protein deficit disease (unknown disease | ABCA9 | 1.0096 | | 1.129464 | | 0.983138 | 0.068041 |
| entity) | | | | | | | |
| Membrane protein deficit disease (unknown disease | CD164 | 1.0714 | 0.2815 | 1.490251 | | 1.00249 | 0.32764 |
| entity) | | | | | | | |
| Membrane protein deficit disease (unknown disease | SLC11A2 | 1.0227 | 0.0428 | 1.05269 | 0.098208 | 1.056032 | 0.026566 |
| entity) | | | | | | | |
| MPS I | IDUA | 1.0369 | 0.2193 | 1.279373 | 0.35447 | 1.395165 | 0.316644 |
| MPS IIIA | SGSH | 1.0275 | 0.3028 | 1.188007 | 0.095157 | 1.370299 | 0.245804 |
| MPS IIIB | NAGLU | 1.0035 | 0.1669 | 1.361794 | 0.16993 | 1.603786 | 0.502552 |
| MPS IIID | GNS | 1.0801 | 0.0757 | 1.24817 | 0.029913 | 1.169007 | 0.159009 |
| Degradation of GAG/GM2 gangliosidosis type I | HEXA | 1.278 | 0.2768 | 1.254903 | 0.114248 | 1.38344 | 0.210797 |
| Degradation of GAG | HYAL3 | 0.8042 | 0.245 | | | 1.709449 | 0.523685 |
| Pompe Disease | GAA | 0.8612 | 0.2392 | 1.643903 | 0.187209 | 1.553296 | 0.682125 |
| Gaucher disease | GBA | 1.1604 | 0.2229 | 1.453569 | 0.182064 | 1.412866 | 0.433759 |
| Fucosidosis | FUCA1 | 1.1357 | 0.2234 | 1.465155 | 0.350256 | 1.631048 | 0.515631 |
| Alpha- mannosidosis | MAN2B1 | 0.9412 | 0.3301 | 1.14414 | 0.102095 | 1.260493 | 0.436065 |
| Beta- mannosidosis | MANBA | 1.3866 | 0.3901 | 2.314417 | 0.364804 | 2.362808 | 0.780241 |
| Sialosis/sialadenosis/galactosialosis | NEU1 | 1.3826 | 0.1926 | 2.239105 | 0.096246 | 2.870597 | 0.424571 |
| GM2 gangliosidosistype AB | GM2A | 1.1234 | 0.3582 | 1.378218 | 0.137079 | 1.739737 | 0.432425 |
| Farber disease | ASAH1 | 1.3803 | 0.5072 | 1.600998 | 0.09063 | 1.672367 | 0.815099 |
| Niemann-Pick disease type A and B | SMPD1 | 1.0652 | 0.1781 | 1.407672 | 0.190768 | 1.384126 | 0.203436 |
| Niemann-Pick disease C | NPC1 | 1.0535 | 0.1894 | 1.388482 | 0.174847 | 1.386653 | 0.419977 |
| Neuronal ceroid lipofuscinose type II | TPP1 | 1.2546 | 0.1911 | 1.538879 | 0.27087 | 2.005653 | 0.492583 |
| Neuronal ceroid lipofuscinose type V | CLN5 | 1.3695 | 0.3427 | 2.00101 | 0.517906 | 2.46425 | 0.286468 |
| Mucolipidosis II and IIIA and B | GNPTAB | 1.2907 | 0.2856 | | | 1.24186 | 0.470351 |
| Mucolipidosis IV | MCOLN1 | 0.9989 | 0.1474 | 1.190594 | 0.029881 | 1.214528 | 0.369553 |
| Cystinosis | CTNS | 1.3472 | 0.6764 | 1.47644 | | 1.495583 | 0.409779 |
| Mucosulfatidosis | SUMF1 | 1.1436 | 0.0877 | 1.486723 | 0.053478 | 1.63139 | 0.152687 |
| Aspartylglucosaminuria | AGA | 1.258 | 0.2217 | 1.672835 | 0.228257 | 1.886867 | 0.538573 |
| Metachromatic leukodystrophy | ARSA | 1.1359 | 0.1761 | 1.19762 | 0.169489 | 1.321031 | 0.230475 |
| Sulfatosis (unknown disease entity) | ARSG | 0.9298 | 0.3007 | 1.403815 | 0.863146 | 1.219551 | 0.382033 |
| Proteosis (unknown disease entity) | CTSA | 1.1392 | 0.2285 | 1.534713 | 0.26009 | 1.458565 | 0.265488 |
| Proteosis (unknown disease entity) | CTSD | 1.0443 | 0.2197 | 1.373366 | 0.12026 | 1.199103 | 0.391386 |
| Proteosis (unknown disease entity) | CTSF | 1.0688 | 0.1873 | 1.342314 | 0.017224 | 1.675405 | 0.516217 |
| Pycnodysostosis | CTSK | 1.0833 | 0.0393 | 1.232062 | 0.115655 | 1.198836 | 0.17374 |
| Proteosis (unknown disease entity) | CTSO | 1.4084 | 0.2398 | 1.910526 | 0.280482 | 1.592421 | 0.373152 |
| Proteosis (unknown disease entity) | LGMN | 1.1978 | 0.1424 | 1.577076 | 0.168746 | 1.427228 | 0.301595 |
| Phosphatosis (unknown disease entity) | ACP2 | 0.9843 | 0.2444 | 1.120136 | 0.277818 | 1.51985 | 0.189843 |
| Phosphatosis (unknown disease entity) | ACP5 | 1.0628 | 0.2819 | 1.386844 | 0.005137 | 1.597037 | 0.093799 |
| Lipidosis (unknown disease entity) | FVT1 | 1.0665 | 0.1599 | 1.349074 | 0.232104 | 1.266987 | 0.188471 |
| Membrane protein deficit disease. | ABCA9 | 1.2742 | 0.119 | 1.074919 | | 1.133843 | 0.368938 |
| (unknown disease entity) | | | | | | | |
| Membrane protein deficit disease | CD164 | 1.1229 | 0.2015 | 1.788157 | 0.276158 | 1.311883 | 0.597279 |
| (unknown disease entity) | | | | | | | |
| Membrane protein deficit disease | SLC11A2 | 1.0909 | 0.109 | 1.170311 | 0.156377 | 1.423506 | 0.204374 |
| (unknown disease entity) | | | | | | | |
| MPS I | IDUA | 1.1407 | 0.1394 | 1.212394 | 0.035436 | 1.309275 | 0.223 |
| MPS IIIA | SGSH | 1.1596 | 0.1624 | 1.122148 | 0.123312 | 0.929923 | 0.272405 |
| MPS IIIB | NAGLU | 1.1022 | 0.1993 | 1.231164 | 0.034618 | 1.30308 | 0.227395 |
| MPS IIID | GNS | 1.2473 | 0.1684 | 1.495338 | 0.024541 | 1.417824 | 0.125403 |
| Degradation of GAG/GM2 gangliosidosis type I | HEXA | 1.0817 | 0.1854 | 1.474107 | 0.373058 | 1.620908 | 0.392138 |
| Degradation of GAG | HYAL3 | 0.8605 | 0.1153 | 0.949153 | | 1.000508 | 0.129694 |
| Pompe Disease | GAA | 1.4222 | 0.3458 | 1.763188 | 0.483822 | 1.955177 | 0.816788 |
| Gaucher disease | GBA | 1.257 | 0.2124 | 1.467255 | 0.133795 | 1.606084 | 0.202878 |
| Fucosidosis | FUCA1 | 1.3114 | 0.2247 | 1.558106 | 0.0808 | 1.633913 | 0.223327 |
| Alpha- mannosidosis | MAN2B1 | 1.036 | 0.0659 | 1.585718 | 0.263484 | 1.923559 | 0.693395 |
| Beta- mannosidosis | MANBA | 1.6385 | 0.3621 | 2.173341 | 0.407895 | 2.386989 | 0.495085 |
| Sialosis/sialadenosis/galactosialosis | NEU1 | 1.6536 | 0.3698 | 2.258588 | 0.412865 | 2.492243 | 0.329339 |
| GM2 gangliosidosis type AB | GM2A | 1.0452 | 0.3491 | 1.612152 | 0.486207 | 1.535849 | 0.64938 |
| Farber disease | ASAH1 | 1.0802 | 0.1007 | 1.412161 | 0.210908 | 1.520289 | 0.286213 |
| Niemann-Pick disease type A and B | SMPD1 | 1.2419 | 0.1758 | 1.457252 | 0.105517 | 1.673001 | 0.238648 |
| Niemann-Pick disease C | NPC1 | 1.567 | 0.3556 | 1.875204 | 0.264845 | 2.268712 | 0.446557 |
| Neuronal ceroid lipofuscinose type II | TPP1 | 1.2672 | 0.1839 | 1.505417 | 0.157549 | 1.602799 | 0.175836 |
| Neuronal ceroid lipofuscinose type V | CLN5 | 1.3471 | 0.2052 | 1.789895 | 0.167178 | 2.125146 | 0.442509 |
| Mucolipidosis II and IIIA and B | GNPTAB | 1.6393 | 0.2006 | | | 1.871984 | 0.056621 |
| Mucolipidosis IV | MCOLN1 | 1.2919 | 0.1137 | 1.439073 | 0.163219 | 1.481838 | 0.193411 |
| Cystinosis | CTNS | 1.5216 | 0.0469 | 1.576991 | 0.060563 | 1.639984 | 0.186261 |
| Mucosulfatidosis | SUMF1 | 1.2364 | 0.1078 | 1.848751 | 0.203287 | 1.705007 | 0.344103 |
| Aspartylglucosaminuria | AGA | 1.3732 | 0.0712 | 1.864242 | 0.392986 | 2.330271 | 0.144843 |
| Metachroniatic leukodystrophy | ARSA | 1.2835 | 0.2425 | 1.218205 | 0.074239 | 1.526216 | 0.234973 |
| Sulfatosis (unknown disease entity) | ARSG | 1.3921 | 0.296 | 2.488141 | 0.327613 | 1.810239 | 0.499047 |
| Proteosis (unknown disease entity) | CTSA | 1.3019 | 0.1974 | 1.637114 | 0.263772 | 1.570966 | 0.307684 |
| Proteosis (unknown disease entity) | CTSD | 1.3322 | 0.3231 | 1.301943 | 0.133944 | 1.938638 | 0.423265 |
| Proteosis (unknown disease entity) | CTSF | 1.417 | 0.3289 | 1.800312 | 0.210353 | 2.06647 | 0.833385 |
| Pycnodysostosis | CTSK | 1.2621 | 0.2243 | 1.387253 | 0.226049 | 1.540819 | 0.286811 |
| Proteosis (unknown disease entity) | CTSO | 1.2507 | 0.3082 | 1.276245 | 0.190954 | 1.470577 | 0.464658 |
| Proteosis | LGMN | 1.3309 | 0.1012 | 1.610264 | 0.202855 | 1.833202 | 0.335402 |
| Phosphatosis (unknown disease entity) | ACP2 | 1.0451 | 0.1279 | 1.062904 | 0.179379 | 1.147271 | 0.200428 |
| Phosphatosis (unknown disease entity) | ACP5 | 1.6259 | 0.5266 | 1.7507 | 0.084298 | 1.763013 | 0.574622 |
| Lipidosis (unknown disease entity) | FVT1 | 1.2441 | 0.253 | 1.603035 | 0.339214 | 1.421771 | 0.352849 |
| Membrane protein deficit disease. | ABCA9 | 1.3825 | 0.3128 | 1.773089 | 0.162575 | 1.635193 | 0.407543 |
| (unknown disease entity) | | | | | | | |
| Membrane protein deficit disease | CD164 | 0.9618 | 0.1815 | 1.426825 | 0.375189 | 1.281965 | 0.325888 |
| (unknown disease entity) | | | | | | | |
| Membrane protein deficit disease | SLC11A2 | 1.2196 | 0.0773 | 1.549296 | 0.294636 | 1.499179 | 0.113596 |
| (unknown disease entity) | | | | | | | |

The group of lysosomal storage disease includes int.al. all disease entities listed in the Table 1 below for which the use of microarray technology allowed authors to identify the effect of genistein on expression of genes encoding appropriate enzymes, which absence or deficit is responsible for individual LSD. Moreover, Table 1 provides genes encoding enzymes from biosynthesis pathway of organic substances accumulated in cells of patients with LSD and enzymes, which modify these compounds, whose expression was found to be modulated in cells exposed to genistein.

Table 1. Selected genes encoding enzymes from biosynthesis pathways of organic substances accumulated in cells of patients with LSD and enzymes which modify these compounds, for which the transcriptomic profiling with the use of DNA microarrays allowed to determine the reduced expression. Selected disease entities belonging to LSD, for which the transcriptomic profiling with the use of DNA microarrays allowed to determine increased expression of genes from the degradation pathways of certain organic compounds.

| Lysosomal disease/Metabolic pathway | Enzyme | Gene |
|---|---|---|
| GAG biosynthesis | N-acetylgalactosamine sulfotransferase | CHST14 |
| GAG biosynthesis | glucosamine sulfotransferase•3A1 | HS3ST3A1 |
| GAG biosynthesis | N-acetylglucosamine transferase | EXT1 |
| GAG biosynthesis | xylosyltransferase | XYLT1 |
| GAG biosynthesis | alpha-sialyltransferase 2 | ST3GAL2 |
| Sphingolipid biosynthesis | alpha-sialyltransferase 4 | ST3GAL4 |
| Sphingolipid biosynthesis | alpha-sialyltransferase 6 | ST3GAL6 |
| MPS I | alpha-L-iduronidase | IDUA |
| MPS IIIA | N-sulfoglucosamine sulfohydrolase | SGSH |
| MPS IIIB | alpha-D-N-acetylglucosaminidase | NAGLU |
| MPS IIID | alpha-N-acetylglucosamine-6-sulphate | GNS |
| | sulfatase | |
| Degradation of GAG | beta-hexosaminidase A | HEXA |
| Degradation of GAG | hyaluronglucosaminidase 3 | HYAL3 |
| Pompe Disease | alpha-glucosidase | GAA |
| Gaucher disease | beta-glucosidase | GBA |
| Fucosidosis | alpha-fucosidase | FUCA1 |
| Alpha-mannosidosis | alpha-mannosidase | MAN2B1 |
| Beta-mannosidosis | beta-mannosidase | MANBA |
| Sialosis/Sialadenosis/Galactosialosis | sialidase1 | NEU1 |
| GM2 gangliosidosis type AB | GM2 activator | GM2A |
| GM2 gangliosidosis type 1 (Tay-Sach | beta-hexosaminidase A | HEXA |
| disease) | | |
| Farber Disease | N-acylsphingosine aminohydrolase | ASAH1 |
| Niemann-Pick disease type A and B | sphingomyelin phosphodiesterase 1 | SMPD1 |
| Niemann-Pick disease type C | Niemann-Pick protein C1 | NPC1 |
| Neuronal ceroid lipofuscinose type II | tripeptidyl peptidase 1 | TPP1 |
| Neuronal ceroid lipofuscinose type V | neuronal ceroid lipofuscinose protein 5 | CLN5 |
| Mucolipidosis II and IIIA and B | UDP-N-acetylglucosamine-1- | GNPTAB |
| | phosphotransferase | |
| Mucolipidosis type IV | mucolipin 1 | MCOLN1 |
| Cystinosis | cystinosin | CTNS |
| Mucosulfatidosis | sulfatase modifying factor 1 | SUMF1 |
| Aspartylglucosaminuria | aspartylglucosaminidase | AGA |
| Metachromatic leukodystrophy | arylsulfatase A | ARSA |
| Sulfatasis (unknown disease entity) | arylsulfatase G | ARSG |
| Proteosis (unknown disease entity) | cathepsin A | CTSA |
| Proteosis (unknown disease entity) | cathepsin D | CTSD |
| Proteosis (unknown disease entity) | cathepsin F | CTSF |
| Pycnodysostosis | cathepsin K | CTSK |
| Proteosis (unknown disease entity) | cathepsin O | CTSO |
| Proteosis (unknown disease entity) | legumin | LGMN |
| Phosphatosis (unknown disease entity) | acid phosphatase 2 | ACP2 |
| Phosphatosis (unknown disease entity) | acid phosphatase 5 | ACP5 |
| Lipidosis (unknown disease entity) | hydrosphingosine reductase | FVT1 |
| Diseases caused by deficit of membrane | factor 9 of subfamily A (ABC1) of proteins | ABCA9 |
| protein associated with lysosomes | containing an ATP binding cassette | |
| (unknown disease entity) | | |
| Diseases caused by deficit of membrane | sialomucin | CD164 |
| protein associated with lysosomes | | |
| (unknown disease entity) | | |
| Diseases caused by deficit of membrane | proton-dependent divalent metal transporter | SLC11A2 |
| protein associated with lysosomes | | |
| (unknown disease entity) | | |

The comparison of the amount of transcripts in the cells cultured in the presence and absence of genistein allowed to indicate particular genes from the pathway of synthesis and degradation of specific substrates i.e. genes, which are involved in synthesis of these cell components as well as genes, which dysfunction is responsible for the occurrence of particular lysosomal disorder. Transcriptomic profiling of these genes using DNA microarrays allowed to indicate the reduced (genes of synthesis) or increased (genes of degradation) expression. This indicates the direct effect of genistein on the regulation of the genes expression, products of which are involved in metabolism of particular substrates.

The results of the studies on the implementation of the concept of non-enzymatic therapy with the use of genistein in the treatment of lysosomal storage disease, especially these with neurological symptoms, lead to obtaining relevant information about mechanisms of gene expression regulation in cells, but could also lead to the development of potential treatment methods for the whole range of lysosomal storage diseases, as per the table above. To make this possible, the inventors focus on investigating genistein's mechanism of action and, on verifying (on the molecular level) the extent of expression modulation by genistein not only of the selected genes, but all human genome sequences known to date. The data obtained on the basis of DNA arrays analysis showed expression of which genes, within the whole cell genome, is modulated by genistein. Based on the previous literature reports related to the phenomenon of transcription factor EB (TFEB) translocation from cytoplasm to nucleus, resulting in an elevation of many lysosomal genes expression [Sardiello et al., 2009; Settembre et al., 2011], the authors were analysing level of TFEB gene expression in cells treated with genistein in attempt to explain the mechanism of genistein action. The results of the research allowed for the conclusion that there are significant differences between the level of TFEB transcripts in the test sample to the control sample, in comparison to reference genes GAPDH and TBP, which show constant expression levels. Thus, the authors of the invention propound the following hypothesis: in the cells treated with genistein the blockage of EGF tyrosine kinase receptor activity occurs, which results in inhibition of cellular signalling cascade [Jakobkieiwcz-Banecka et al., 2009] and contributes to the increased TFEB activity and its translocation from cytoplasm to nucleus as a result of overproduction of this protein due to increased expression of the gene encoding it. In turn, localisation of TFEB in the nucleus is responsible for elevation of expression of multiple lysosomal genes and increase in the rate of lysosomal exocytosis and degradation of organic substances accumulating in cells of patients with LSD.

In summary, results of the inventors allowed to indicate the use of genistein to reduce the level of organic substances accumulated in lysosomal storage diseases, and to indicate LSDs, which could be potentially treated with the use of genistein (i.e. by reduced expression of genes involved in synthesis and/or by increased expression of genes involved in degradation of particular substrate accumulated in cells of patients with LSD).

1. Genistein for use in non-enzymatic method of treatment and/or prevention of the lysosomal storage diseases (LSDs) i.e. diseases with the underlying defect in degradation and resulting accumulation of organic compounds in lysosomes, to reduce the level of storage of organic compounds by reducing the rate of efficiency of accumulating organic substances synthesis and/or by increasing the rate of efficiency of cellular deposits degradation.

2. Genistein for use according to claim 1, wherein the increase of the efficiency of degradation of the organic substances accumulated in the cells of patients with LSD exposed to genistein, due to the modulation of expression of genes encoding one or more of the enzymes involved in this process, occurs by the overproduction of the transcription factor EB (TFEB) due to increased expression of its gene.

3. Genistein for use according to claim 1, wherein the decrease of the efficiency of synthesis of the organic substances accumulated in the cells of patients with LSD exposed to genistein is due to the modulation of expression of genes encoding one or more of the enzymes involved in this process.

4. Genistein for use according to claim 1, wherein there is a modulation of expression of genes involved in the metabolism of storage substances (i.e. synthesis and degradation) i.e. genes encoding for enzymes of the biosynthesis pathway of organic substances accumulating in cells of patients with LSD and enzymes, which are modifying these compounds, as well as lysosomal enzymes.

5. Genistein for use according to claim 1, wherein gene expression is monitored with the use of transcriptomic methods.

6. Genistein for use according to claim 1, wherein DNA microarrays are used.

7. Genistein for use according to claim 1, wherein the impaired enzymatic activity of lysosomes relates to enzymes selected from alpha-L-iduronidase, N-sulfoglucosamine sulfohydrolase, alpha-D-N-acetylglucosaminidase, alpha-N-acetylglucosamine-6-sulphate sulfatase, beta-hexosaminidase A, hyaluronglucosaminidase 3, alpha-glucosidase, beta-glucosidase, alpha-fucosidase, alpha-mannosidase, beta-mannosidase, sialidase 1, GM2 activator, beta-hexosaminidase A, N-acylsphingosine aminohydrolase, sphingomyelin phosphodiesterase 1, Niemann-Pick C1 protein, tripeptidyl peptidase 1, neuronal ceroid lipofuscinose protein 5, UDP-N-acetylglucosamine-1-phosphotransferase, mucolipin 1, cystinosin, sulfatase modifying factor 1, aspartylglucosaminidase, arylsulfatase A, arylsulfatase G, cathepsin A, cathepsin D, cathepsin F, cathepsin K, cathepsin O, legumin, acid phosphtase 2, acid phosphtase 5, hydrosphingosine reductase, factor 9 of subfamily A (ABC 1) of proteins containing an ATP binding cassette, sialomucins, proton-dependent divalent metal transporter. Furthermore, the use according to claim 1, wherein the reduced level of biosynthesis of organic substances accumulated in the cells of patients with LSD and modifying these compounds relates to enzymes selected from: N-acetylgalactosamine sulfotransferase, glucosamine sulfotransferase 3A1, N-acetylglucosamine transferase, xylosyltransferase and alpha-sialyltransferases 2, 4 and 6.

8. Genistein for use according to claim 1, wherein the disease entity is selected from MPS I, MPS IIIA, MPS IIIB, MPS IIID, Pompe disease, Gaucher disease, fucosidosis, alpha-mannosidosis, beta-mannosidosis, sialosis/sialadenosis/galactosialosis, GM2 gangliosidosis type AB, GM2 gangliosidosis type I (Tay-Sachs disease), Farber disease, Niemann-Pick disease type A and B, Niemann-Pick disease type C, neuronal ceroid lipofuscinose type II, neuronal ceroid lipofuscinose type V, mucolipidosis type II and III A and B, mucolipidosis type IV, cystinosis, mucosulfatidosis, aspartylglucosoaminuria, metachromatic leukodystrophy, pycnodysostosis and other selected disease entities from lysosomal storage diseases.
